(19) United States
(12) Patent Application Publication
(10) Pub. No.: US 2021/0219914 A1
(43) Pub. Date: Jul. 22, 2021

(54) Monitoring Earphone Intended for Autism Spectrum Disorder

(71) Applicant: Innovation Sound Technology Co., Ltd., Shenzhen (CN)

(72) Inventors: Shun Ming Yuen, Shenzhen (CN); Wai Yin Mung, Shenzhen (CN); Ka Ming Wu, Shenzhen (CN)

(21) Appl. No.: 17/225,061

(22) Filed: Apr. 7, 2021

Related U.S. Application Data
(63) Continuation of application No. PCT/CN2019/083632, filed on Apr. 22, 2019.

(30) Foreign Application Priority Data
Feb. 27, 2019 (CN) 201920245910.4

(51) Int. Cl. A61B 5/00; H04R 1/10; A61B 5/16; A61B 5/372

(52) U.S. Cl. CPC A61B 5/6817; H04R 1/105; H04R 1/1016; H04R 1/1033; H04R 2420/07; A61B 5/16; A61B 5/372; A61B 5/0006; A61B 5/002; H04R 1/1041

(57) ABSTRACT

The application belongs to the field of earphone technology, and discloses a type of monitoring earphones intended for autism spectrum disorder. The upper part of headband is fixed with an electroencephalogram signal sensor, and the two sides thereof are respectively secured with No. 1 buckle and No. 2 buckle, which are used to fix No. 1 No. 1 signal processor and No. 2 signal processor on the upper part of the headband. Once the user is found by the application to be not himself/herself, the earphone will play his/her favorite music with Bluetooth, serving the function of music therapy.

### CROSS REFERENCE TO RELATED APPLICATIONS

The present application is a Continuation Application of PCT Application No. PCT/CN2019/083632 filed on Apr. 22, 2019, which claims the benefit of Chinese Patent Application No. 201920245910.4 filed on Feb. 27, 2019. All the above are hereby incorporated by reference.

### FIELD OF THE INVENTION

The application specifically relates to a type of monitoring earphones intended for autism spectrum disorder, which belongs to the field of earphone technology.

### BACKGROUND OF THE INVENTION

The characteristics of autism include social and communication disorders, repetitive behaviors, resistance to routine changes in daily life, and unusual reactions to sensory experiences (such as sound, light, smell, etc.). Part of patients may develop self-harming behaviors, emotional problems, intellectual disability, hyperactivity, or epilepsy at the same time. Studies have shown that children with ASD are more sensitive to their auditory stimuli than average persons, so children with ASD have been found to use self-treatment strategies, including covering their ears, crying and even self-harm. When they arrive in an unfamiliar environment, they would produce great reactions and abnormal behaviors, which affects their daily life, social life and learning. Research has also revealed that some music with special rhythm or melody can effectively soothe their emotions, so as to disperse the influence of the environment on them.

At present, there are no earphones on the market with the function of treating autism, which is urgent to be improved.

### CONTENT OF THE APPLICATION

The technical problem to be solved by the application is to overcome the existing defects and to provide a monitoring earphone for autism spectrum disorder. Collecting user data by setting up an electroencephalogram signal sensor, once the user is found to be not himself/herself, the earphone will play his/her favorite music via Bluetooth, serving the function of music therapy. Playing music through Bluetooth can help users improve their psychological and physiological problems. Hence, the earphone may also be used for meditation experience. Meditation can exercise one's mental concentration, adjust one's own breathing and thoughts, help a patient achieve a state of synchronization of body and mind, reduce stress, control anxiety, and improve sleep quality, etc. The application can effectively solve the problems in the background of the invention.

In order to solve the above technical problems, the application provides the following technical solutions:

The application provides a type of monitoring earphones intended for autism spectrum disorder, comprising No. 1 signal processor, No. 1 buckle, a headband, No. 2 buckle, and No. 2 signal processor, an electroencephalogram signal sensor, wires and earphones. The upper part of the headband is fixed with an electroencephalogram signal sensor, and the two sides thereof are respectively secured with No. 1 buckle and No. 2 buckle, which are used to fix No. 1 signal processor and No. 2 signal processor on the upper part of the headband, and to facilitate changes in the location of the No. 1 signal processor and the No. 2 signal processor on the upper part of the headband. The No. 1 signal processor (1) and the No. 2 signal processor (5) are connected to the earphone through wires.

The output end of the electroencephalogram signal sensor is respectively connected with the input ends of the No. 1 signal processor and the No. 2 signal processor.

As a preferred technical solution of the application, the No. 1 signal processor and the No. 2 signal processor are connected to the earphone through wires.

As a preferred technical solution of the application, the upper part of the No. 1 signal processor and the No. 2 signal processor is provided with hangers.

As a preferred technical solution of the application, batteries are fixed inside the No. 1 signal processor and the No. 2 signal processor.

As a preferred technical solution of the application, the input ends of the No. 1 signal processor and the No. 2 signal processor are electrically connected to the output end of the Bluetooth module.

Beneficial effects achieved by the application: It collects user data by setting up an electroencephalogram signal sensor. Once the user is found to be not himself/herself, the earphone will play his/her favorite music via Bluetooth, serving the function of music therapy. Playing music through Bluetooth can help users improve their psychological and physiological problems. Hence, the earphone may also be used for meditation experience. Meditation can exercise one's mental concentration, adjust one's own breathing and thoughts, help a patient achieve a state of synchronization of body and mind, reduce stress, control anxiety, and improve sleep quality, etc.

### BRIEF INTRODUCTION OF THE DRAWINGS

The accompanying drawings are used to provide a further understanding of the application and constitute a part of the Specification. Together with the embodiments of the application, they are intended to interpret the application but do not constitute a limitation to the application.

In the accompanying drawings:

FIG. 1 is a schematic diagram for the overall structure of a monitoring earphone for autism spectrum disorder according to embodiments of the application;

FIG. 2 is a partial structural diagram of a monitoring earphone for autism spectrum disorder according to embodiments of the application;

Numbers in the drawings: 1. No. 1 signal processor; 2. No. 1 buckle; 3. Headband; 4. No. 2 buckle; 5. No. 2 signal processor; 6. Electroencephalogram signal sensor; 7. Wires; 8. Earphones.

### DETAILED DESCRIPTION OF THE PREFERRED EMBODIMENTS

The preferred embodiments of the application are described below in conjunction with the drawings. It shall be understood that the preferred embodiments described herein are only used to illustrate and interpret the application, rather than to limit the application.

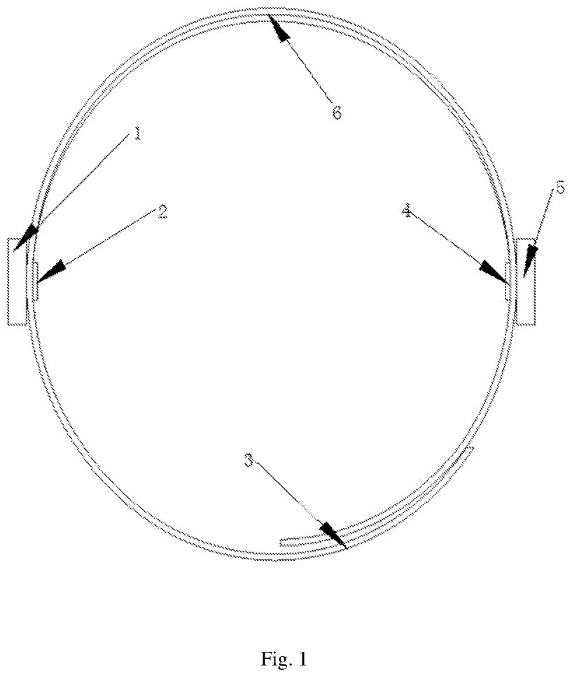

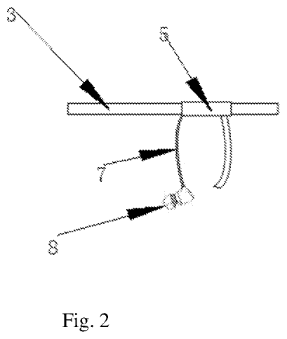

Embodiments: Please refer to FIG. 1-2. The application provides a type of monitoring earphones intended for autism spectrum disorder, comprising No. 1 signal processor 1, No. 1 buckle 2, a headband 3, No. 2 buckle 4, and No. 2 signal processor 5, an electroencephalogram signal sensor 6, wires 7 and earphones 8. The upper part of the headband 3 is fixed with an electroencephalogram signal sensor 6, and the two sides thereof are respectively secured with the No. 1 buckle 2 and the No. 2 buckle 4, which are used to fix the No. 1 signal processor 1 and the No. 2 signal processor 5 on the upper part of the headband 3, and to facilitate changes in the location of the No. 1 signal processor 1 and the No. 2 signal processor 5 on the upper part of the headband 3. The No. 1 signal processor 1 and the No. 2 signal processor 5 are connected to the earphones 8 through wires 7. The output end of the electroencephalogram signal sensor 6 is respectively connected with the input ends of the No. 1 signal processor 1 and the No. 2 signal processor 5.

Further, the No. 1 signal processor 1 and the No. 2 signal processor 5 are connected to the earphones 8 through wires 7.

Further, the upper part of the No. 1 signal processor 1 and the No. 2 signal processor 5 are fitted with hangers.

Further, batteries are fixed inside the No. 1 signal processor 1 and the No. 2 signal processor 5.

Further, the input ends of the No. 1 signal processor 1 and the No. 2 signal processor 5 are electrically connected to the output end of the Bluetooth module.

It should be noted that the application, being a type of monitoring earphones intended for autism spectrum disorder, collects user data by setting up an electroencephalogram signal sensor. Once the user is found to be not himself/herself, the earphone will play his/her favorite music via Bluetooth, serving the function of music therapy. Playing music through Bluetooth can help users improve their psychological and physiological problems. Hence, the earphone may also be used for meditation experience. Meditation can exercise one's mental concentration, adjust one's own breathing and thoughts, help a patient achieve a state of synchronization of body and mind, reduce stress, control anxiety, and improve sleep quality, etc.

Last thing to note: The above descriptions are only preferred embodiments of the present invention, and are not used to limit the application. Although the application has been described in detail with reference to the foregoing embodiments, those skilled in the art may still modify the technical solutions described in the foregoing embodiments, or equivalently replace some of the technical features. Any modification, equivalent replacement, improvement, etc. made within the spirit and principle of the application shall be included in the protection scope of the application.

What is claimed is:

1. A type of monitoring earphones intended for autism spectrum disorder, wherein it comprises No. 1 signal processor (1), No. 1 buckle (2), a headband (3), No. 2 buckle B (4), No. 2 signal processor (5), an electroencephalogram signal sensor (6), wires (7) and earphones (8); the upper part of the headband (3) is fixed with an electroencephalogram signal sensor (6), and the two sides thereof are respectively secured with No. 1 buckle (2) and No. 2 buckle (5), which are used to fix the No. 1 signal processor (1) and the No. 2 signal processor (5) on the upper part of the headband (3), and to facilitate changes in the location of the No. 1 signal processor (1) and the No. 2 signal processor (5) on the upper part of the headband (3); the No. 1 signal processor (1) and the No. 2 signal processor (5) are connected to the earphones (8) via wires (7); the output end of the electroencephalogram signal sensor (6) is respectively connected with the input ends of the No. 1 signal processor (1) and the No. 2 signal processor (5).

2. A monitoring earphone intended for autism spectrum disorder according to claim 1, wherein the No. 1 signal processor (1) and the No. 2 signal processor (5) are connected to the earphone (8) through wires (7).

3. A monitoring earphone intended for autism spectrum disorder according to claim 1, wherein the upper parts of the No. 1 signal processor (1) and the No. 2 signal processor (5) are provided with hangers.

4. A monitoring earphone intended for autism spectrum disorder according to claim 1, wherein batteries are fixed inside the No. 1 signal processor (1) and the No. 2 signal processor (5).

5. A monitoring earphone intended for autism spectrum disorder according to claim 1, wherein the input ends of the No. 1 signal processor (1) and the No. 2 signal processor (5) are electrically connected with the output end of a Bluetooth module.
